(19) United States
(12) Patent Application Publication
(10) Pub. No.: US 2026/0144444 A1
(43) Pub. Date: May 28, 2026

(54) DETECTION DEVICE AND METHOD FOR MANUFACTURING DETECTION DEVICE

(71) Applicant: Japan Display Inc., Tokyo (JP)

(72) Inventor: Ken OHARA, Tokyo (JP)

(21) Appl. No.: 19/451,887

(22) Filed: Jan. 16, 2026

Related U.S. Application Data
(63) Continuation of application No. PCT/JP2024/024109, filed on Jul. 3, 2024.

(30) Foreign Application Priority Data
Jul. 24, 2023 (JP) 2023-120200

(51) Int. Cl. A61B 5/00

(52) U.S. Cl. CPC A61B 5/0059; A61B 5/6826; A61B 2562/0233; A61B 2562/046; A61B 2562/12

(57) ABSTRACT

According to an aspect, a detection device includes: a first substrate; a plurality of organic photodiodes each including a lower electrode, a lower buffer layer, an active layer, an upper buffer layer, and an upper electrode, which are stacked on the first substrate; a detection area in which the organic photodiodes are arranged; a sealing film that covers the detection area; and a plurality of first openings that are provided so as to penetrate at least from the sealing film to the first substrate.

### CROSS-REFERENCE TO RELATED APPLICATION

This application claims the benefit of priority from Japanese Patent Application No. 2023-120200 filed on July 24, 2023 and International Patent Application No. PCT/JP2024/024109 filed on July 3, 2024, the entire contents of which are incorporated herein by reference.

### BACKGROUND

### Technical Field

What is disclosed herein relates to a detection device and a method for manufacturing a detection device.

### Description of the Related Art

Input display devices (detection devices) that can be worn at a fixed position adjacent to the skin for a long time are known (for example, Japanese Patent Application Laid-open Publication No. 2009-032005).

In such input display devices (detection devices) that can be worn adjacent to the skin for a long time, a portion adjacent to the skin may become moist due to sweating because of being used while being worn on the skin for a long time.

For the foregoing reasons, there is a need for a detection device and a method for manufacturing a detection device capable of improving ventilation to reduce moisture due to sweating.

### SUMMARY

According to an aspect, a detection device includes: a first substrate; a plurality of organic photodiodes each including a lower electrode, a lower buffer layer, an active layer, an upper buffer layer, and an upper electrode, which are stacked on the first substrate; a detection area in which the organic photodiodes are arranged; a sealing film that covers the detection area; and a plurality of first openings that are provided so as to penetrate at least from the sealing film to the first substrate.

According to an aspect, a method for manufacturing a detection device includes: stacking a circuit forming layer, an organic photodiode, and a sealing film that covers the organic photodiode on one surface of a first substrate in the order as listed; forming, after the stacking, a first opening so as to penetrate from the sealing film to the first substrate in a direction orthogonal to the first substrate; stacking a second substrate on the sealing film after the forming; and heating the second substrate after the stacking the second substrate.

### BRIEF DESCRIPTION OF THE DRAWINGS

FIG. 1A is a schematic view illustrating an exemplary external appearance when a finger is accommodated inside a detection device according to a first embodiment as viewed from a lateral side of a housing;

FIG. 1B is a schematic view illustrating an exemplary external appearance when a human body is accommodated inside a detection device according to the first embodiment as viewed from the lateral side of the housing;

FIG. 1C is a schematic view illustrating an exemplary external appearance when a human body wears a detection device according to the first embodiment as viewed from the lateral side of the housing;

FIG. 2 is a schematic sectional view of a section taken along II-II' illustrated in FIG. 1A;

FIG. 3 is a plan view schematically illustrating the detection device according to the first embodiment;

FIG. 4 is a block diagram illustrating a configuration example of the detection device according to the first embodiment;

FIG. 5 is a circuit diagram illustrating the detection device according to the first embodiment;

FIG. 6 is a plan view schematically illustrating a plurality of pixels and first openings;

FIG. 7 is a sectional view taken along VII-VII' in FIG. 6;

FIG. 8 is a magnified schematic configuration view of a sensor;

FIG. 9 is a sectional view taken along IX-IX' in FIG. 8;

FIG. 10 is a sectional view taken along X-X' in FIG. 8;

FIG. 11 is a schematic sectional view of a detection device according to a first modification of the first embodiment;

FIG. 12 is a schematic sectional view of a detection device according to a second modification of the first embodiment;

FIG. 13 is a schematic sectional view of a detection device according to a third modification of the first embodiment;

FIG. 14 is a plan view schematically illustrating the pixels and the first openings of a detection device according to a second embodiment;

FIG. 15 is a magnified schematic configuration view of the sensor according to the second embodiment;

FIG. 16 is a plan view schematically illustrating the pixels and first openings of a detection device according to a third embodiment;

FIG. 17 is a plan view schematically illustrating a magnified view of a periphery of the first openings in FIG. 16;

FIG. 18 is a sectional view taken along XVIII-XVIII' in FIG. 16;

FIG. 19 is a schematic sectional view of a detection device according to a first modification of the third embodiment;

FIG. 20 is a schematic sectional view of a detection device according to a second modification of the third embodiment;

FIG. 21 is a schematic sectional view of a detection device according to a third modification of the third embodiment;

FIG. 22 is a plan view schematically illustrating the pixels, the first openings, and second openings of a detection device according to a fourth embodiment;

FIG. 23 is a sectional view taken along XXIII-XXIII' in FIG. 22; and

FIG. 24 is an explanatory diagram for explaining a manufacturing method according to a fifth embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### First Embodiment

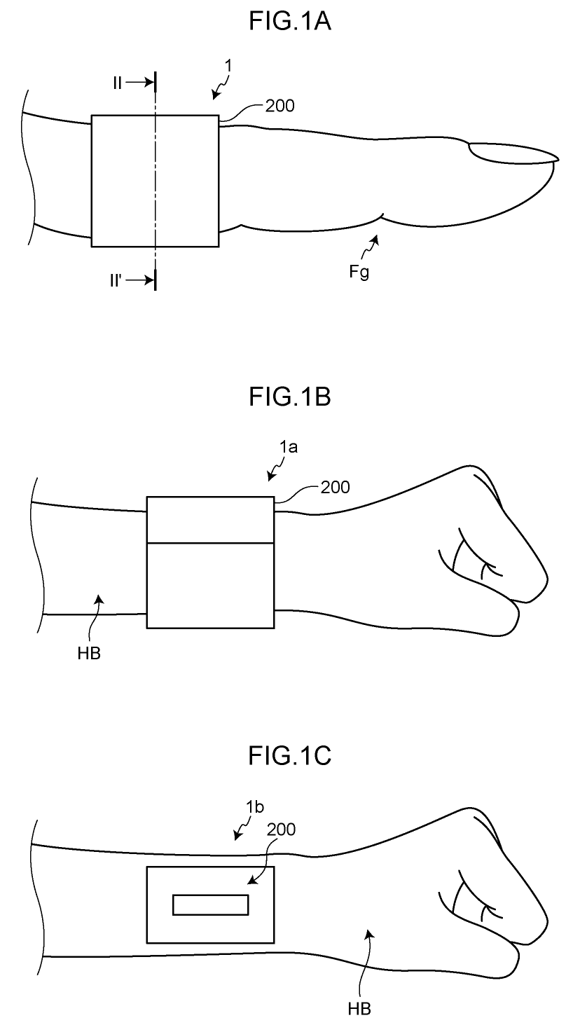

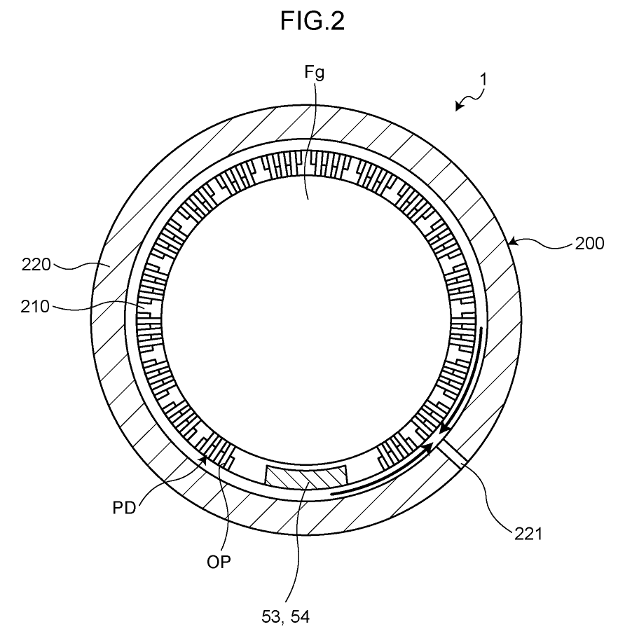

FIG. 1A is a schematic view illustrating an exemplary external appearance when a finger is accommodated inside a detection device according to a first embodiment as viewed from a lateral side of a housing. FIG. 2 is a schematic sectional view of a section taken along II-II' illustrated in FIG. 1A.

A detection device 1 illustrated in FIG. 1A is a finger ring-shaped device that can be worn on and removed from a human body and is worn on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body, and is an object to be measured. The detection device 1 is made into a finger ring or a wristband so as to be easily carried by a user. In the following description, the detection device 1 is assumed to be used as a finger ring.

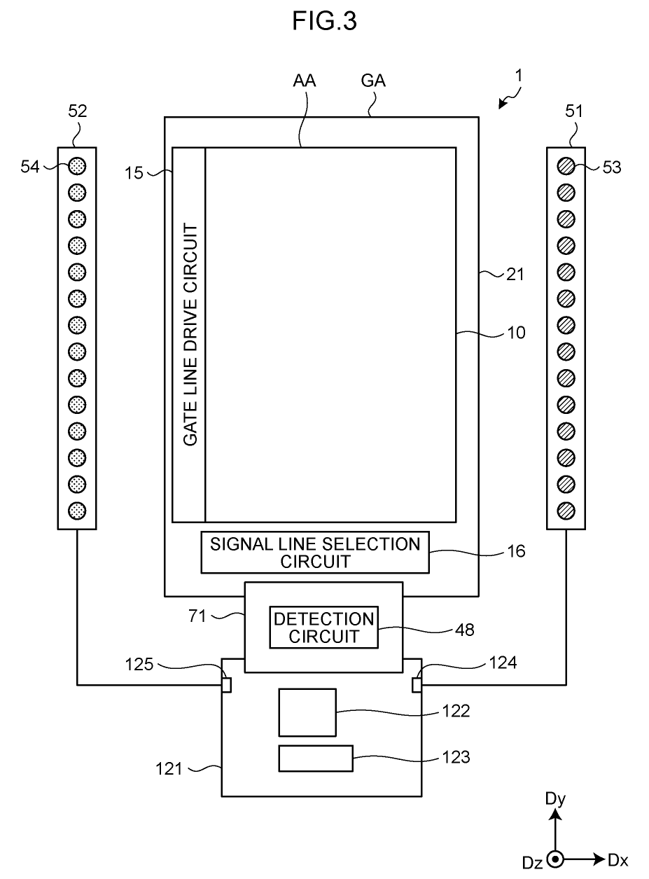

As illustrated in FIG. 2, the detection device 1 includes a housing 200, light sources 53 and 54, and an organic photodiode PD (organic optical sensor). The detection device 1 is a device that includes a battery (not illustrated) inside the housing 200 and is operated by power of the battery. FIG. 2 does not illustrate components (for example, various substrates such as a first substrate 21 (refer to FIG. 3)) other than the housing 200, the organic photodiode PD, and the light sources 53 and 54.

The housing 200 is a mounting member that is formed in a ring shape (annular shape) wearable on the finger Fg, and is worn on the living body. In the example illustrated in FIG. 2, the housing 200 includes a sealing film 210 and an exterior portion 220. The housing 200 is formed in the ring shape by integrating the sealing film 210 with the exterior portion 220. The sealing film 210 accommodates therein the light sources 53 and 54, the organic photodiode PD, and other components. The sealing film 210 is formed in a ring shape using a housing material, such as a light-transmitting synthetic resin or silicon. The exterior portion 220 has a surface of the housing 200 that covers the sealing film 210. The exterior portion 220 is formed in a ring shape using a member of, for example, a metal, a non-light-transmitting synthetic resin, or the like. The exterior portion 220 has a ventilation opening 221.

FIG. 1B is a schematic view illustrating an exemplary external appearance when a human body is accommodated inside a detection device 1a according to the first embodiment as viewed from the lateral side of the housing. The detection device 1a illustrated in FIG. 1B is a ring-shaped device that can be worn on and removed from a human body HB, and is worn on an arm of the human body HB. The detection device 1 illustrated in FIG. 1B is, for example, a smartwatch, a wristwatch, or a wristband. The human body HB is the body of the person to be authenticated whose identity is to be verified by the detection device 1a, and examples thereof include, but are not limited to, wrists, arms, and legs. The detection device 1a can detect the biometric information on the living body from the human body HB wearing the detection device 1.

FIG. 1C is a schematic view illustrating an exemplary external appearance when a human body wears a detection device 1b according to the first embodiment is viewed from the lateral side of the housing. The detection device 1b illustrated in FIG. 1C is an adhesive plaster-like device that can be worn on and removed from the human body HB, and can be worn on a surface of the human body HB by being attached to or placed on the surface of the human body HB. The detection device 1b can detect the biometric information on the living body from the human body HB wearing the detection device 1.

In FIG. 2, each of a plurality of first openings OP is provided so as to penetrate the sealing film 210, the organic photodiode PD, and the first substrate 21 (refer to FIG. 3) in the normal direction.

The ventilation opening 221 is provided in a penetrating manner in the normal direction in order to discharge sweat generated from the finger Fg to the outside. The sweat is discharged to the outside through the ventilation opening 221 via any of the first openings OP.

A plurality of the organic photodiodes PD detect the biometric information on the living body from an object to be detected, such as the finger Fg. Specifically, light emitted from light sources 53 and 54 and transmitted or reflected by the object to be detected, such as the finger Fg, irradiates the organic photodiodes PD.

The organic photodiodes PD detect light emitted by the light sources 53 and 54 and reflected by the finger Fg or the like, light directly incident thereon, and other light.

The light sources 53 and 54 are provided at locations that do not overlap the organic photodiodes PD inside the sealing film 210 of the housing 200, and are configured to be capable of emitting light toward the object to be detected, such as the finger Fg, wearing the ring-shaped housing 200. The locations of the light sources 53 and 54 illustrated in FIG. 2 are merely exemplary, and can be changed as appropriate. That is, the light sources 53 and 54 can be located in any positions as long as the light emitted from the light sources 53 and 54 and transmitted or reflected by the object to be detected, such as the finger Fg, irradiates the organic photodiodes PD properly.

FIG. 3 is a plan view schematically illustrating the detection device according to the first embodiment. As illustrated in FIG. 3, the detection device 1 includes the first substrate 21, a sensor 10, a gate line drive circuit 15, a signal line selection circuit 16, a detection circuit 48, a control circuit 122, a power supply circuit 123, a first light source base member 51, a second light source base member 52, and light sources 53 and 54. The first light source base member 51 is provided with a plurality of the light sources 53. The second light source base member 52 is provided with a plurality of the light sources 54.

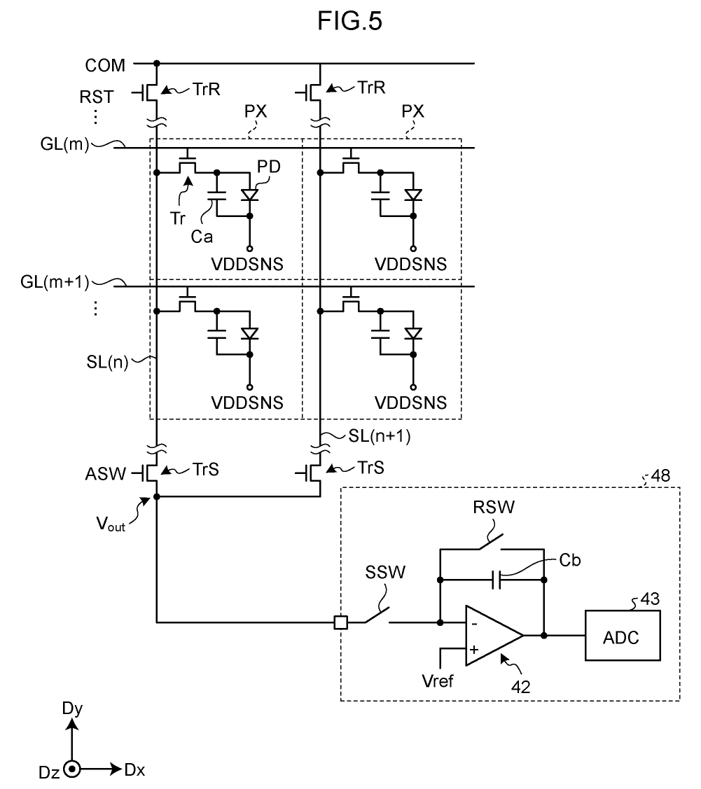

The first substrate 21 is electrically coupled to a control substrate 121 via a wiring substrate 71. The wiring substrate 71 is, for example, a flexible printed circuit board or a rigid circuit board. The wiring substrate 71 is provided with the detection circuit 48. The control substrate 121 is provided with the control circuit 122 and the power supply circuit 123. The control circuit 122 is a field-programmable gate array (FPGA), for example. The control circuit 122 supplies control signals to the sensor 10, the gate line drive circuit 15, and the signal line selection circuit 16 to control detection operations of the sensor 10. The control circuit 122 also supplies control signals to the light sources 53 and 54 to control lighting and non-lighting of the light sources 53 and 54. The power supply circuit 123 supplies voltage signals, such as a sensor power supply signal (sensor power supply voltage) VDDSNS (refer to FIG. 5), to the sensor 10, the gate line drive circuit 15, and the signal line selection circuit 16. The power supply circuit 123 also supplies a power supply voltage to the light sources 53 and 54.

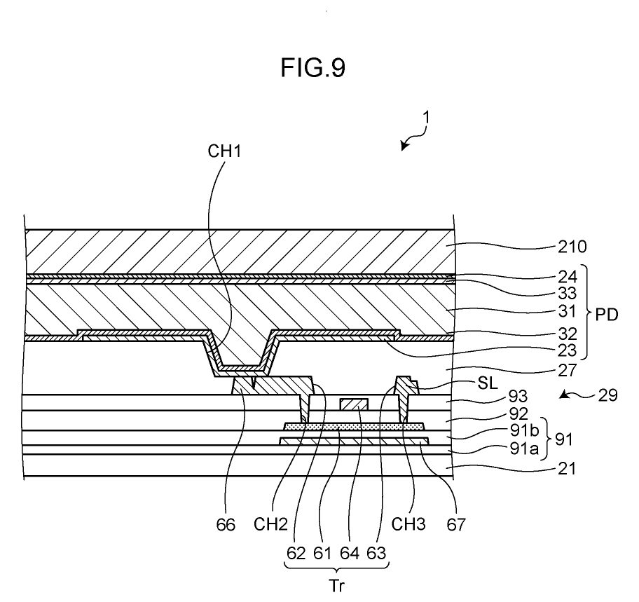

The first substrate 21 has a detection area AA and a peripheral area GA. The detection area AA is an area in which the organic photodiodes PD (refer to FIG. 9) included in the sensor 10 are arranged. The peripheral area GA is an area between the outer perimeter of the detection area AA and the outer edges of the first substrate 21, and is an area not provided with the organic photodiodes PD.

The gate line drive circuit 15 and the signal line selection circuit 16 are provided in the peripheral area GA. Specifically, the gate line drive circuit 15 is provided in an area extending along a second direction Dy in the peripheral area GA. The signal line selection circuit 16 is provided in an area extending along a first direction Dx in the peripheral area GA, and is provided between the sensor 10 and the detection circuit 48.

In the following description, the first direction Dx is one direction in a plane parallel to the first substrate 21. The second direction Dy is one direction in the plane parallel to the first substrate 21 and is a direction orthogonal to the first direction Dx. The second direction Dy may non-orthogonally intersect the first direction Dx.

A third direction Dz is a direction orthogonal to the first direction Dx and the second direction Dy and is a direction normal to a principal surface of the first substrate 21. The term "plan view" refers to a positional relation when viewed from a direction orthogonal to the first substrate 21.

The light sources 53 are provided on the first light source base member 51, and arranged along the second direction Dy. The light sources 54 are provided on the second light source base member 52, and arranged along the second direction Dy. The first light source base member 51 and the second light source base member 52 are electrically coupled to the control circuit 122 and the power supply circuit 123, through respective terminals 124 and 125 provided on the control substrate 121.

For example, inorganic light-emitting diodes (LEDs) or organic electroluminescent (EL) diodes (organic light-emitting diodes (OLEDs)) are used as the light sources 53 and 54. The light sources 53 and 54 emit light having wavelengths different from each other. The light sources 53 and 54 include a plurality of light sources so as to be capable of emitting near-infrared light, red light, and green light.

First light emitted from the light sources 53 is reflected on a surface of the object to be detected, such as the finger Fg, and enters the sensor 10. As a result, the sensor 10 can detect a fingerprint by detecting a shape of asperities on the surface of the finger Fg or the like. Second light emitted from the light sources 54 is reflected in the finger Fg or the like, or transmitted through the finger or the like, and enters the sensor 10. As a result, the sensor 10 can detect information on the living body in the finger Fg or the like. Examples of the information on the living body include, but are not limited to, pulse waves, pulsation, and a vascular image of the finger or a palm. That is, the detection device 1 may be configured as a fingerprint detection device to detect a fingerprint or a vein detection device to detect a vascular pattern of, for example, veins.

The arrangement of the light sources 53 and 54 illustrated in FIG. 3 is merely exemplary and can be changed as appropriate. The detection device 1 is provided with a plurality of types of the light sources 53 and 54 as light sources. However, the light sources are not limited thereto, and may be of one type. For example, the light sources 53 and 54 may be arranged on each of the first and the second light source base members 51 and 52. The light sources 53 and 54 may be provided on one light source base member, or three or more light source base members. Alternatively, only at least one light source needs to be disposed.

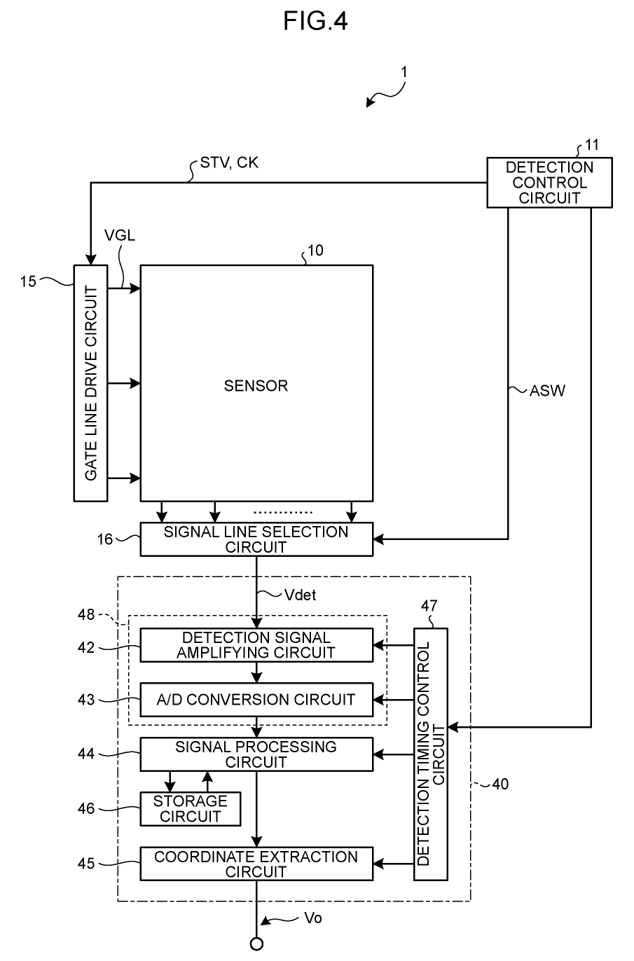

FIG. 4 is a block diagram illustrating a configuration example of the detection device according to the first embodiment. As illustrated in FIG. 4, the detection device 1 further includes a detection control circuit 11 and a detector (detection signal processing circuit) 40. The control circuit 122 includes one, some, or all functions of the detection control circuit 11. The control circuit 122 also includes one, some, or all functions of the detector 40 other than those of the detection circuit 48.

The sensor 10 includes the organic photodiodes PD. Each of the organic photodiodes PD included in the sensor 10 outputs an electrical signal corresponding to the light emitted thereto as a detection signal Vdet to the signal line selection circuit 16. The sensor 10 performs the detection in response to a gate drive signal VGL supplied from the gate line drive circuit 15.

The detection control circuit 11 supplies respective control signals to the gate line drive circuit 15, the signal line selection circuit 16, and the detector 40 to control operations of these components. The detection control circuit 11 supplies various control signals including, for example, a start signal STV and a clock signal CK to the gate line drive circuit 15. The detection control circuit 11 also supplies various control signals including, for example, a selection signal ASW to the signal line selection circuit 16. The detection control circuit 11 also supplies various control signals to the light sources 53 and 54 to control the lighting and the non-lighting of each group of the light sources 53 and 54.

The gate line drive circuit 15 drives a plurality of gate lines GL (refer to FIG. 5) based on various control signals. The gate line drive circuit 15 sequentially or simultaneously selects the gate lines GL, and supplies the gate drive signal VGL to the selected gate lines GL. Through this operation, the gate line drive circuit 15 selects the organic photodiodes PD coupled to the gate lines GL.

The signal line selection circuit 16 includes a switch circuit that sequentially or simultaneously selects a plurality of signal lines SL (refer to FIG. 5). The signal line selection circuit 16 is a multiplexer, for example. The signal line selection circuit 16 couples the selected signal lines SL to the detection circuit 48 based on the selection signal ASW supplied from the detection control circuit 11. Through this operation, the signal line selection circuit 16 outputs the detection signals Vdet of the organic photodiodes PD to the detector 40.

The detector 40 includes the detection circuit 48, a signal processing circuit 44, a coordinate extraction circuit 45, a storage circuit 46, and a detection timing control circuit 47. The detection timing control circuit 47 controls the detection circuit 48, the signal processing circuit 44, and the coordinate extraction circuit 45 to operate those circuits synchronously based on a control signal supplied from the detection control circuit 11.

The detection circuit 48 is an analog front-end (AFE) circuit, for example. The detection circuit 48 is a signal processing circuit having functions of at least a detection signal amplifying circuit 42 and an analog-to-digital (A/D) conversion circuit 43. The detection signal amplifying circuit 42 amplifies the detection signals Vdet. The A/D conversion circuit 43 converts analog signals output from the detection signal amplifying circuit 42 into digital signals.

The signal processing circuit 44 detects predetermined physical quantities received by the sensor 10 based on output signals of the detection circuit 48. The signal processing circuit 44 is a logic circuit. The signal processing circuit 44 can detect the asperities on the surface of the finger or the palm based on the signals from the detection circuit 48 when the finger is in contact with or in proximity to a detection surface. The signal processing circuit 44 can also detect the information on the living body based on the signals from the detection circuit 48. Examples of the information on the living body include, but are not limited to, the vascular image, the pulse waves, the pulsation, and a blood oxygen level of the finger or the palm.

The storage circuit 46 temporarily stores therein signals calculated by the signal processing circuit 44. The storage circuit 46 may be, for example, a random-access memory (RAM) or a register circuit.

The coordinate extraction circuit 45 obtains detected coordinates of the asperities on the surface of the finger or the like when the contact or proximity of the finger is detected by the signal processing circuit 44. The coordinate extraction circuit 45 also obtains detected coordinates of blood vessels in the finger or the palm. The coordinate extraction circuit 45 is a logic circuit. The coordinate extraction circuit 45 combines the detection signals Vdet output from the respective organic photodiodes PD of the sensor 10 to generate two-dimensional information indicating the shape of the asperities on the surface of the finger or the like and two-dimensional information indicating the shape of the blood vessels in the finger or the palm. The coordinate extraction circuit 45 may output the detection signals Vdet as sensor output voltages Vo instead of calculating the detected coordinates.

FIG. 5 is a circuit diagram illustrating the detection device according to the first embodiment. FIG. 5 also illustrates a circuit configuration of the detection circuit 48. As illustrated in FIG. 5, a sensor pixel PX includes the organic photodiode PD, a capacitive element Ca, and a drive transistor Tr. The capacitive element Ca is capacitance (sensor capacitance) generated in the organic photodiode PD and is equivalently coupled in parallel to the organic photodiode PD.

FIG. 5 illustrates two gate lines GL(m) and GL(m+1) arranged in the second direction Dy among the gate lines GL. FIG. 5 also illustrates two signal lines SL(n) and SL(n+1) arranged in the first direction Dx among the signal lines SL. The sensor pixel PX is an area surrounded by the gate lines GL and the signal lines SL.

The drive transistors Tr are provided correspondingly to the organic photodiodes PD. Each of the drive transistors Tr is configured with a thin-film transistor, and in this example, configured with an n-channel metal- oxide-semiconductor (MOS) thin-film transistor (TFT).

Each of the gate lines GL is coupled to the gates of the drive transistors Tr arranged in the first direction Dx. Each of the signal lines SL is coupled to either the sources or the drains of the drive transistors Tr arranged in the second direction Dy. The other of the sources and the drains of the drive transistors Tr are each coupled to the anode of the organic photodiode PD and the capacitive element Ca.

The cathode of the organic photodiode PD is supplied with the sensor power supply signal VDDSNS from the power supply circuit 123 (refer to FIG. 3). The signal line SL and the capacitive element Ca are supplied with a sensor reference voltage COM serving as an initial potential of the signal line SL and the capacitive element Ca from the power supply circuit 123 via a reset transistor TrR.

When the sensor pixel PX is irradiated with light in an exposure period, a current corresponding to the amount of the light flows through the organic photodiode PD. As a result, an electric charge is stored in the capacitive element Ca. When the drive transistor Tr is turned on in a readout period, a current corresponding to the electric charge stored in the capacitive element Ca flows through the signal line SL. The signal line SL is coupled to the detection circuit 48 via an output transistor TrS of the signal line selection circuit 16. Thus, the detection device 1 can detect a signal corresponding to the intensity of the light irradiating the organic photodiode PD for each of the sensor pixels PX.

During the readout period, a switch SSW is turned on to couple the detection circuit 48 to the signal line SL. The detection signal amplifying circuit 42 of the detection circuit 48 converts a current or an electric charge supplied from the signal line SL into a voltage corresponding thereto. A reference potential (Vref) having a fixed potential is supplied to a non-inverting input portion (+) of the detection signal amplifying circuit 42, and the signal line SL is coupled to an inverting input portion (-) of the detection signal amplifying circuit 42. In the present embodiment, the same signal as the sensor reference voltage COM is supplied as the reference potential (Vref) voltage. The control circuit 122 (refer to FIG. 3) calculates, as each of the sensor output voltages Vo, the difference between the detection signal Vdet when light irradiates the organic photodiode PD and the detection signal Vdet when light does not irradiate the organic photodiode PD. The detection signal amplifying circuit 42 includes a capacitive element Cb and a reset switch RSW. During a reset period, the reset switch RSW is turned on to reset the electric charge of the capacitive element Cb.

The drive transistor Tr is not limited to the n-type TFT, and may be configured with a p-type TFT. The pixel circuit of the sensor pixel PX illustrated in FIG. 5 is merely exemplary. The sensor pixel PX may be provided with a plurality of transistors corresponding to one organic photodiode PD.

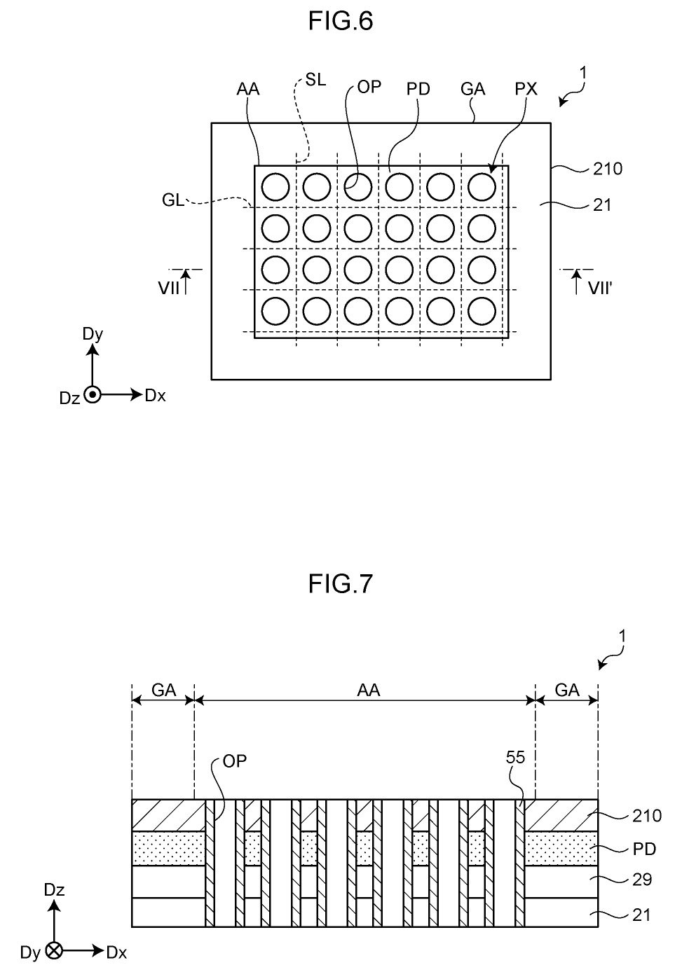

FIG. 6 is a plan view schematically illustrating the pixels and the first openings. As illustrated in FIG. 6, the sensor pixels PX are arranged in a matrix having a row-column configuration in the detection area AA of the first substrate 21 and each include the organic photodiode PD. In FIG. 6, the sensor pixels PX are arranged in four rows and six columns. FIG. 6, however, illustrates the arrangement of the sensor pixels PX in a simplified manner for ease of understanding. The detection device 1 may include a larger number of the sensor pixels PX in five or more rows and seven or more columns, depending on the type of the object to be detected and the resolution of the detection.

The gate lines GL each extend in the first direction Dx, and are arranged with gaps interposed therebetween in the second direction Dy. The signal lines SL each extend in the second direction Dy, and are arranged with gaps interposed therebetween in the first direction Dx. The sensor pixels PX (organic photodiodes PD) are provided in areas surrounded by the adjacent gate lines GL and the adjacent signal lines SL.

The sealing film 210 is provided so as to cover the detection area AA including the sensor pixels PX (organic photodiodes PD). The detection device 1 has the first openings OP in the detection area AA. The first openings OP are provided in areas overlapping the respective sensor pixels PX, and are arranged in a matrix having a row-column configuration in the detection area AA. In other words, each of the first openings OP is provided in an area surrounded by two of the gate lines GL and two of the signal lines SL.

The first openings OP are circular in plan view. The areas (diameters) of the first openings OP are substantially equal to one another. In the present embodiment, the areas (diameters) of the first openings OP are all equal, but some of them may be different.

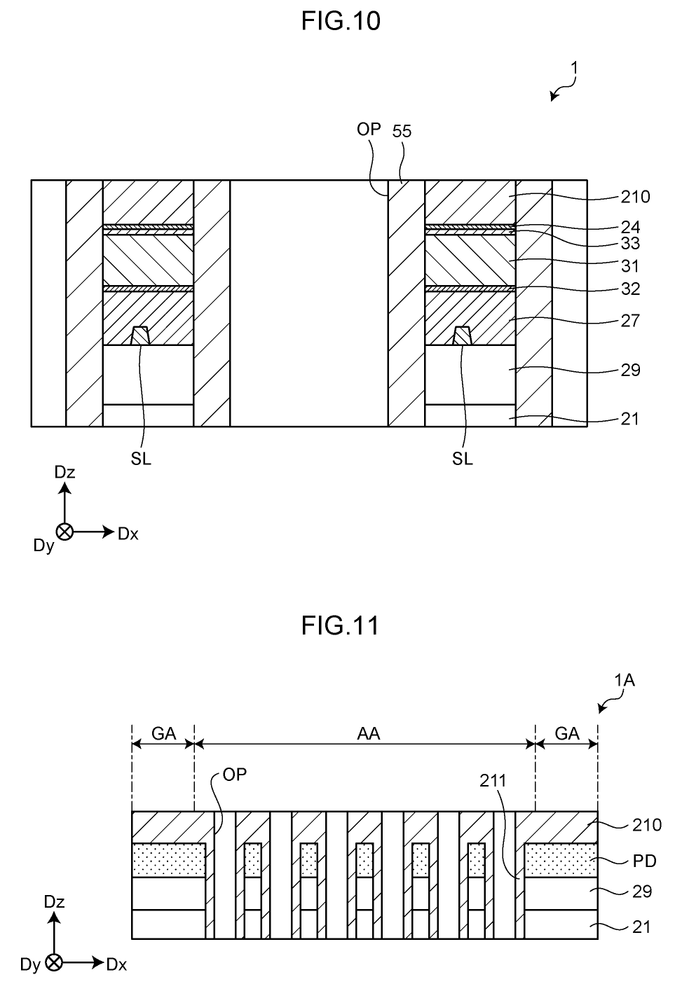

FIG. 7 is a sectional view taken along VII-VII' in FIG. 6. FIG. 7 illustrates a configuration of layers in a simplified manner for ease of understanding. A detailed multilayered configuration of the organic photodiodes PD will be described later with reference to FIG. 9. A detailed multilayered configuration of a circuit forming layer 29 will be described later with reference to FIG. 10.

In the following description, a direction from the first substrate 21 toward the sealing film 210 in a direction orthogonal to a surface of the first substrate 21 is referred to as "upper side" or simply "above". A direction from the sealing film 210 toward the first substrate 21 is referred to as "lower side" or simply "below".

As illustrated in FIG. 7, in the detection device 1, the circuit forming layer 29, the organic photodiodes PD, and the sealing film 210 are stacked in this order on the first substrate 21. The circuit forming layer 29 is a layer that is provided on the first substrate 21, and in which various transistors, such as the drive transistors Tr, and various types of wiring, such as the gate lines GL and the signal lines SL, illustrated in FIG. 5 are formed.

The organic photodiodes PD are provided on the circuit forming layer 29. The organic photodiodes PD are provided continuously over the detection area AA and the peripheral area GA. The organic photodiodes PD provided in the peripheral area GA are configured so as not to serve as optical sensors.

The sealing film 210 is provided on the organic photodiodes PD. The first openings OP are provided so as to penetrate from the sealing film 210 to the first substrate 21 in the third direction Dz. In the present embodiment, the first openings OP are provided in areas overlapping the detection area AA and not provided in areas overlapping the peripheral area GA.

The side wall of each of the first openings OP is covered with a resin film 55. The resin film 55 is formed, for example, of polyethylene terephthalate (PET) that is a film-like synthetic resin. The resin film 55 is shaped to match the shape of the side wall of the first opening OP, and the shape thereof is not limited and may have a curved surface or a quadrilateral shape. This configuration can seal the organic photodiodes PD, the circuit forming layer 29, and the first substrate 21.

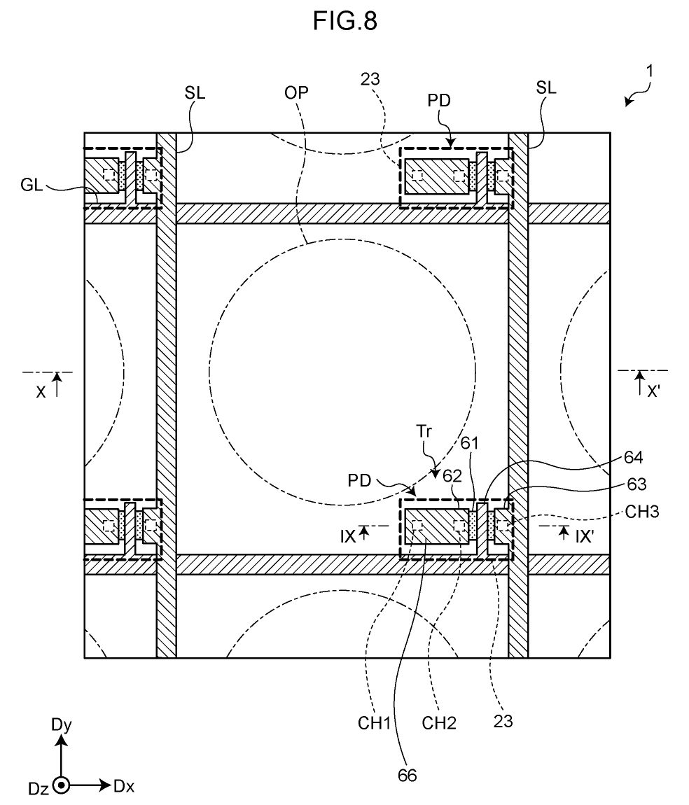

The following describes a detailed configuration of the organic photodiodes PD. FIG. 8 is a magnified schematic configuration view of the sensor. For ease of viewing, FIG. 8 illustrates the first openings OP with alternate long and short dashed lines.

As illustrated in FIG. 8, the detection device 1 includes the organic photodiodes PD provided on the first substrate 21. The right and bottom sides of a lower electrode 23 overlap portions of the signal line SL and the gate line GL, respectively. The left and top sides of the lower electrode 23 are provided at locations that do not overlap the signal line SL and the gate line GL, respectively.

Each of the first openings OP is provided in an area not provided with the lower electrode 23 in plan view. This configuration does not expose the lower electrode 23 in the first opening OP, thus ensuring the reliability.

The drive transistor Tr includes a semiconductor layer 61, a source electrode 62, a drain electrode 63, and a gate electrode 64. The semiconductor layer 61 extends along the gate line GL and intersects the gate electrode 64 in plan view. The gate electrode 64 is coupled to the gate line GL, and extends in a direction (second direction Dy) orthogonal to the gate line GL.

One end of the semiconductor layer 61 is coupled to the source electrode 62 through a contact hole CH2. The source electrode 62 is coupled to a coupling pad 66 and drawn to a central portion of the lower electrode 23. The lower electrode 23 is coupled to the coupling pad 66 at the central portion through a contact hole CH1. With this configuration, the source electrode 62 of the drive transistor Tr is electrically coupled to the organic photodiode PD. The other end of the semiconductor layer 61 is coupled to the drain electrode 63 through a contact hole CH3. The drain electrode 63 is coupled to the signal line SL.

The shape, the arrangement pitch, and other factors of the lower electrodes 23 illustrated in FIG. 8 are merely exemplary, and can be changed as appropriate depending on the characteristics and detection accuracy required for the detection device 1.

FIG. 9 is a sectional view taken along IX-IX' in FIG. 8. FIG. 10 is a sectional view taken along X-X' in FIG. 8. As illustrated in FIG. 9, in the detection device 1, the circuit forming layer 29, an insulating film 27, the organic photodiode PD, and the sealing film 210 are stacked in this order on the first substrate 21. The first substrate 21 is an insulating substrate and is made using, for example, a glass substrate of quartz, alkali-free glass, or the like. The first substrate 21 is not limited to a flat plate-shaped substrate and may have a curved surface. In this case, the first substrate 21 is formed in a strip shape, for example, by polyethylene terephthalate (PET) that is a film-like synthetic resin.

The circuit forming layer 29 is provided on the first substrate 21. The insulating film 27 is provided on the circuit forming layer 29 including the drive transistor Tr so as to cover the signal line SL. The insulating film 27 is an organic planarizing film formed of an organic insulating material.

As illustrated in FIG. 9, the circuit forming layer 29 includes an undercoat film 91, a gate insulating film 92, and an interlayer insulating film 93 as insulating films.

The undercoat film 91 is, for example, a two-layer multilayered structure that includes insulating films 91a and 91b. The undercoat film 91 is formed of an inorganic insulating film, such as a silicon nitride film or a silicon oxide film, for example. The configuration of the undercoat film 91 is not limited to that illustrated in FIG. 9. For example, the undercoat film 91 may be a single-layer film or a multilayered film of three or more layers.

A light-blocking film 67 is provided on the insulating film 91a. The light-blocking film 67 is provided between the semiconductor layer 61 and the first substrate 21. The light-blocking film 67 can reduce light penetrating from the first substrate 21 side into a channel region of the semiconductor layer 61.

The drive transistor Tr is provided on the first substrate 21. The semiconductor layer 61 is provided on the undercoat film 91. The gate insulating film 92 is provided on the undercoat film 91 so as to cover the semiconductor layer 61. The gate insulating film 92 is an inorganic insulating film, such as a silicon oxide film. The gate electrode 64 is provided on the gate insulating film 92.

In the present embodiment, the drive transistor Tr has a top-gate structure. The drive transistor Tr is not limited thereto. The drive transistor Tr may have a bottom-gate structure, or a dual-gate structure with the gate electrodes 64 provided on both the top and bottom sides of the semiconductor layer 61.

The interlayer insulating film 93 is provided on the gate insulating film 92 so as to cover the gate electrode 64. The interlayer insulating film 93 has a multilayered structure of a silicon nitride film and a silicon oxide film, for example. The source electrode 62 and the drain electrode 63 are provided on the interlayer insulating film 93. The source electrode 62 is coupled to a source region of the semiconductor layer 61 through the contact hole CH2 provided in the gate insulating film 92 and the interlayer insulating film 93. The drain electrode 63 is coupled to a drain region of the semiconductor layer 61 through the contact hole CH3 provided in the gate insulating film 92 and the interlayer insulating film 93.

The contact hole CH1 is provided in the lower electrode 23 so as to penetrate the insulating film 27 in the thickness direction (third direction Dz). The lower electrode 23 is coupled to the coupling pad 66 at the bottom of the contact hole CH1.

The insulating film 27 is provided on the interlayer insulating film 93 so as to cover the source electrode 62 and the drain electrode 63 of the drive transistor Tr. In the present embodiment, the contact hole CH1 in the insulating film 27 is provided in an area overlapping the source electrode 62.

The organic photodiode PD is provided on the insulating film 27. The organic photodiode PD includes the lower electrode 23, a lower buffer layer 32, an active layer 31, an upper buffer layer 33, and an upper electrode 24. In the organic photodiode PD, the lower electrode 23, the lower buffer layer 32, the active layer 31, the upper buffer layer 33, and the upper electrode 24 are stacked in this order. The organic photodiode PD of the present embodiment is an organic photodiode (OPD) that uses an organic semiconductor as the active layer 31.

The lower electrode 23 is an anode electrode of the organic photodiode PD and is formed, for example, of a light-transmitting conductive material such as indium tin oxide (ITO). The lower electrodes 23 are separated from one another so as to correspond to the organic photodiodes PD. The lower buffer layer 32, the active layer 31, the upper buffer layer 33, and the upper electrode 24 are provided continuously across the organic photodiodes PD. Specifically, the lower buffer layer 32, the active layer 31, the upper buffer layer 33, and the upper electrode 24 are provided so as to overlap the lower electrodes 23 of the organic photodiodes PD that are adjacent to each other. The lower electrode 23 is electrically coupled to the source electrode 62 at the bottom of the contact hole CH1 near the drive transistor Tr. The lower buffer layer 32, the active layer 31, the upper buffer layer 33, and the upper electrode 24 may be separated for each of the sensor pixels PX.

The active layer 31 changes in characteristics (for example, voltage-current characteristics and resistance value) depending on light emitted thereto. An organic material is used as a material of the active layer 31. Specifically, the active layer 31 has a bulk heterostructure containing a mixture of a p-type organic semiconductor and an n-type fullerene derivative ((6,6)-phenyl-C₆₁-butyric acid methyl ester (PCBM)) that is an n- type organic semiconductor. As the active layer 31, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 31 can be formed by a vapor deposition process (dry process) using any of the low-molecular-weight organic materials listed above. In this case, the active layer 31 may be, for example, a multilayered film of CuPc and F₁₆CuPc, or a multilayered film of rubrene and C₆₀. The active layer 31 can also be formed by a coating process (wet process). In this case, the active layer 31 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 31 can be a film made of a mixture of P3HT and PCBM, or a film made of a mixture of F8BT and PDI.

The lower buffer layer 32 is a hole transport layer, and the upper buffer layer 33 is an electron transport layer. The lower buffer layer 32 and the upper buffer layer 33 are provided to facilitate holes and electrons generated in the active layer 31 to reach the lower electrode 23 or the upper electrode 24. The lower buffer layer 32 (hole transport layer) is in direct contact with the top of the lower electrode 23 and is also provided on an insulating film 35 between the adjacent lower electrodes 23. The active layer 31 is in direct contact with the top of the lower buffer layer 32. The material of the hole transport layer is a metal oxide layer. For example, tungsten oxide (WO₃) or molybdenum oxide is used as the metal oxide layer.

The upper buffer layer 33 (electron transport layer) is in direct contact with the top of the active layer 31, and the upper electrode 24 is in direct contact with the top of the upper buffer layer 33. Polyethylenimine ethoxylated (PEIE) is used as a material of the electron transport layer.

The materials and the manufacturing methods of the lower buffer layer 32, the active layer 31, and the upper buffer layer 33 are merely exemplary, and other materials and manufacturing methods may be used. For example, each of the lower buffer layer 32 and the upper buffer layer 33 is not limited to a single-layer film, and may be formed as a multilayered film that includes an electron block layer and a hole block layer. The lower buffer layer 32 may be an electron transport layer; the upper buffer layer 33 a hole transport layer; the lower electrode 23 may be a cathode electrode; and the upper electrode 24 may be an anode electrode.

The upper electrode 24 is provided on the upper buffer layer 33. The upper electrode 24 is a cathode electrode of the organic photodiodes PD, and is continuously formed over the entire detection area AA. In other words, the upper electrode 24 is continuously provided in the upper side layer of the organic photodiodes PD. The upper electrode 24 faces the lower electrodes 23 with the lower buffer layer 32, the active layer 31, and the upper buffer layer 33 interposed therebetween. The upper electrode 24 is formed, for example, of a light-transmitting conductive material such as ITO or indium zinc oxide (IZO). The upper electrode 24 may be a multilayered film of a plurality of light-transmitting conductive materials. When the upper electrode 24 is not provided, the upper buffer layer 33 serves also as the upper electrode 24 and becomes the cathode electrode of the photodiode PD.

The sealing film 210 is provided so as to cover the organic photodiodes PD. Specifically, the sealing film 210 is provided on the upper electrode 24. If the sealing film 210 is not provided, the upper electrode 24 also serves as the sealing film 210.

As illustrated in FIG. 10, each of the first openings OP is provided so as to penetrate the sealing film 210, the organic photodiode PD with the lower electrode 23 excluded (lower buffer layer 32, active layer 31, upper buffer layer 33 and upper electrode 24), the insulating film 27, the circuit forming layer 29, and the first substrate 21, in the third direction Dz. In other words, an area not provided with the first openings OP overlaps at least the signal line SL and the gate line GL (refer to FIG. 8).

In the detection device 1 of the present embodiment, the first openings OP are provided in the areas overlapping the detection area AA that contact the finger Fg or the like. As a result, even when the detection device 1 is worn on the skin of the finger Fg or the like and used for a long time, the sweat generated from the finger Fg or the like is discharged through the first openings OP from the ventilation opening 221 to the outside. Therefore, the detection device 1 has improved ventilation. Thus, even when the detection device 1 is worn on the skin of the finger Fg or the like and made adjacent to the skin, the moisture due to sweating is reduced, and the detection device 1 can be used for a long time.

The shape, positions, number, and other factors of the first openings OP illustrated in FIGS. 6 to 10 are merely exemplary, and can be changed as appropriate depending on the arrangement of the organic photodiodes PD and other factors. The configuration of the organic photodiodes PD illustrated in FIGS. 9 and 10 is merely exemplary, and can be changed as appropriate. For example, the upper electrode 24 may be the anode electrode of the organic photodiode PD, and the lower electrode 23 may be the cathode electrode of the organic photodiodes PD.

### First Modification of First Embodiment

FIG. 11 is a schematic sectional view of a detection device according to a first modification of the first embodiment. In the following description, the same components as those in the embodiment described above are denoted by the same reference numerals, and the description thereof will not be repeated.

As illustrated in FIG. 11, in a detection device 1A according to the first modification of the first embodiment, compared with the detection device 1 according to the first embodiment, the side walls of the first openings OP are covered with a material 211 that is the same as the sealing film 210.

As a result, the side walls of the first openings OP can be easily covered with only the sealing film 210, without using a member such as the resin film 55.

### Second Modification of First Embodiment

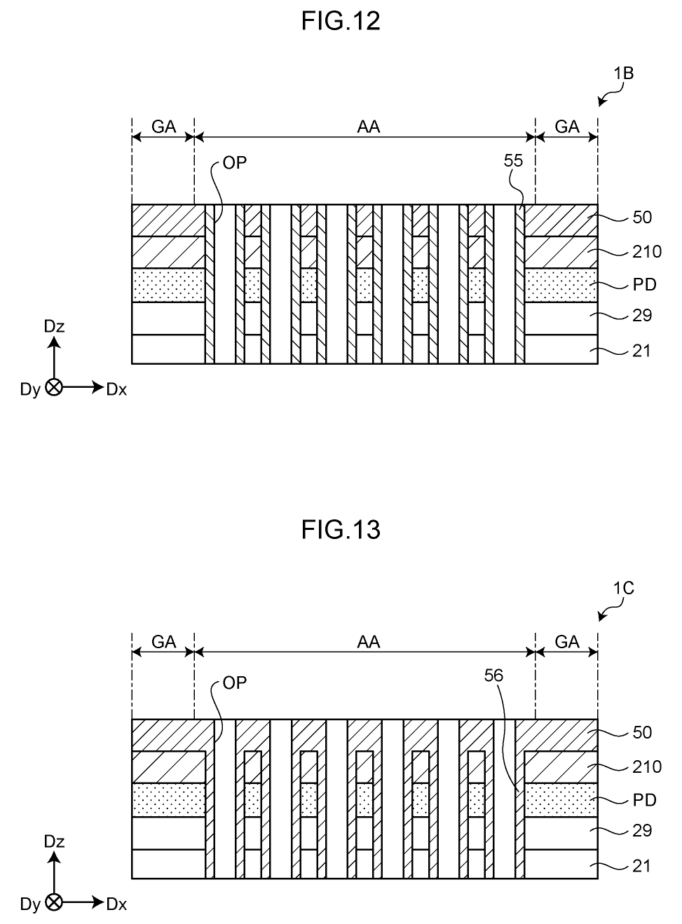

FIG. 12 is a schematic sectional view of a detection device according to a second modification of the first embodiment. In the following description, the same components as those in the embodiment described above are denoted by the same reference numerals, and the description thereof will not be repeated.

As illustrated in FIG. 12, in a detection device 1B according to the second modification of the first embodiment, a second substrate 50 is provided on the sealing film 210, compared with the detection device 1 according to the first embodiment. The first openings OP are provided so as to penetrate from the second substrate 50 to the first substrate 21 in the third direction Dz. The side walls of the first openings OP provided in the second substrate 50 are each covered with the resin film 55.

The second substrate 50 overlaps and faces the first substrate 21 as viewed along the third direction Dz.

In the same way as the first substrate 21, the second substrate 50 is an insulating substrate, and is made using, for example, a glass substrate of quartz, alkali-free glass, or the like. The second substrate 50 is not limited to a flat plate-shaped substrate, and may have a curved surface. In this case, the second substrate 50 is formed in a strip shape, for example, by polyethylene terephthalate (PET) that is a film-like synthetic resin. The second substrate 50 covers the sealing film 210 and is a deformable substrate.

The configuration and operations of the detection device 1B according to the second modification of the first embodiment are substantially the same as those of the detection device 1 of the first embodiment, and therefore, will not be described.

### Third Modification of First Embodiment

FIG. 13 is a schematic sectional view of a detection device according to a third modification of the first embodiment. In the following description, the same components as those in the embodiment described above are denoted by the same reference numerals, and the description thereof will not be repeated.

As illustrated in FIG. 13, in a detection device 1C according to the third modification of the first embodiment, the second substrate 50 is provided on the sealing film 210, compared with the detection device 1 according to the first embodiment. The first openings OP are provided so as to penetrate from the second substrate 50 to the first substrate 21 in the third direction Dz. The side walls of the first openings OP are covered with a material 56 that is the same as the second substrate 50, instead of the resin film 55.

As a result, the side walls of the first openings OP can be easily covered with only the second substrate 50, without using a member such as the resin film 55.

### Second Embodiment

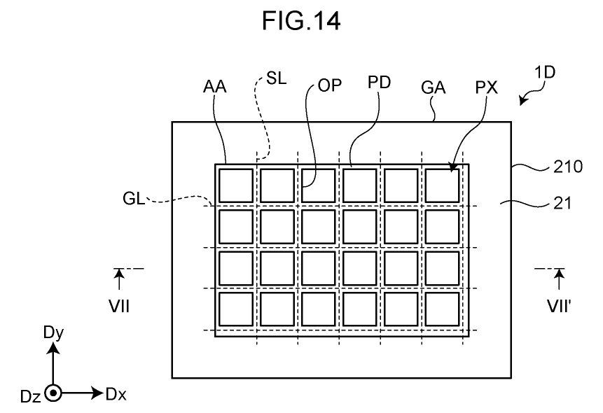

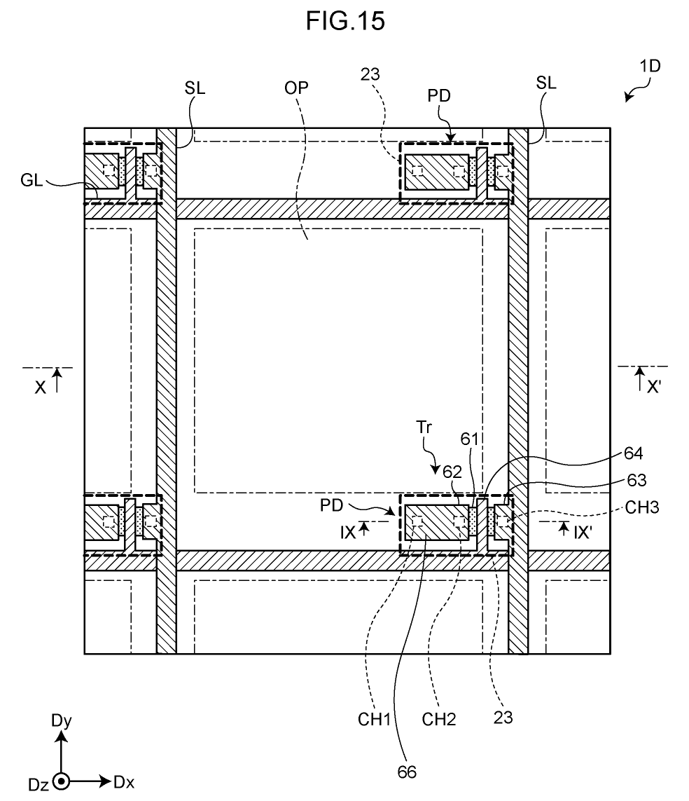

FIG. 14 is a plan view schematically illustrating the pixels and the first openings of a detection device according to a second embodiment. FIG. 15 is a magnified schematic configuration view of the sensor according to the second embodiment. In the following description, the same components as those in the embodiment described above are denoted by the same reference numerals, and the description thereof will not be repeated. A section taken along VII-VII' in FIG. 14 and a section taken along X-X' in FIG. 15 include components denoted by the same reference numerals as those described in the first embodiment.

As illustrated in FIG. 14, in a detection device 1D according to the second embodiment, each of the first openings OP is provided in a substantially quadrilateral shape in the area surrounded by the gate lines GL and the

signal lines SL. In more detail, as illustrated in FIG. 15, the first opening OP extends along each of the gate lines GL and the signal lines SL. The first opening OP is provided in an area not overlapping the electrodes of the drive transistor Tr.

In the present embodiment, the area of the first opening OP in the detection area AA is larger than that in the detection device 1 according to the first embodiment, so that the moisture due to sweating can be reduced more effectively.

### Third Embodiment

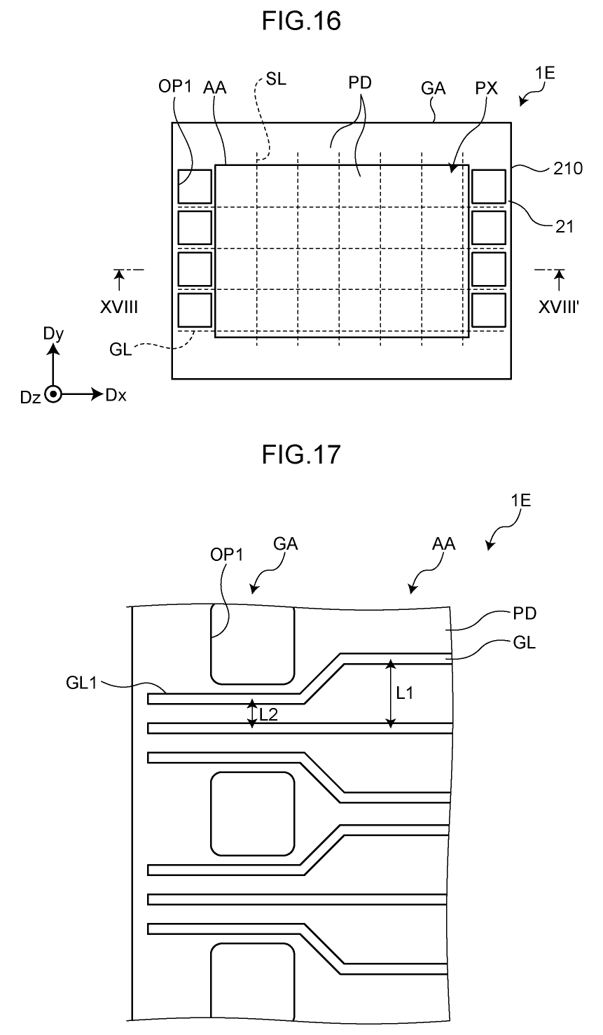

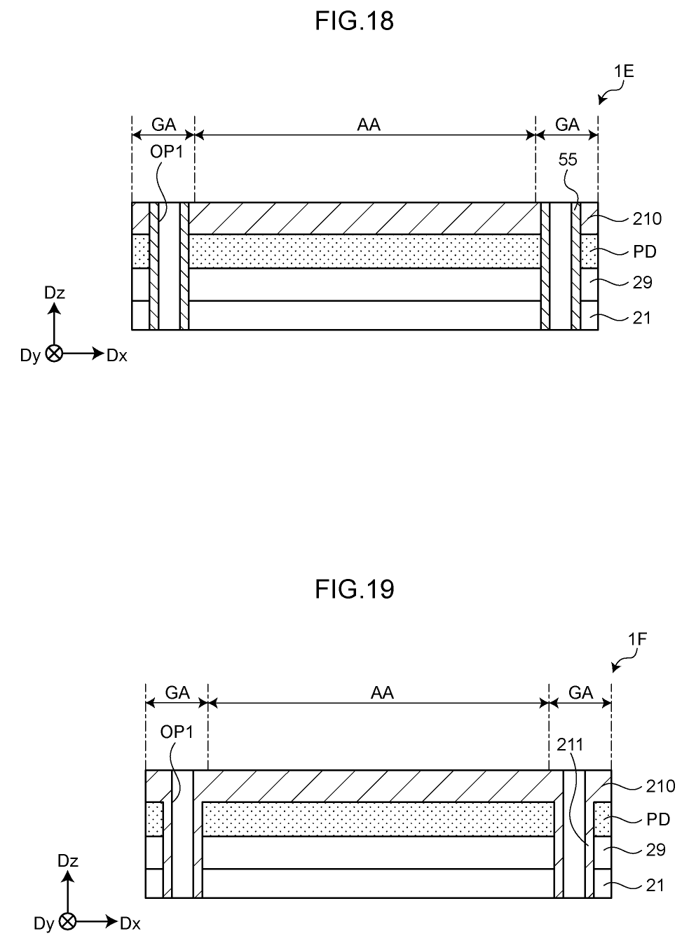

FIG. 16 is a plan view schematically illustrating the pixels and first openings of a detection device according to a third embodiment. FIG. 17 is a plan view schematically illustrating a magnified view of the optical sensors in the peripheral area in FIG. 16. FIG. 18 is a sectional view taken along XVIII-XVIII' in FIG. 16. In the following description, the same components as those in either of the embodiments described above are denoted by the same reference numerals, and the description thereof will not be repeated.

As illustrated in FIG. 16, in a detection device 1E according to the third embodiment, the organic photodiodes PD are provided in the detection area AA and the peripheral area GA of the first substrate 21. A plurality of first openings OP1 are provided along the second direction Dy in areas of the peripheral area GA surrounded by the gate lines GL and the signal lines SL.

As illustrated in FIG. 17, in the detection device 1E, the pitch of a plurality of gate lines GL1 arranged adjacent to the first openings OP1 in the peripheral area GA is smaller than the pitch of the gate lines GL in the detection area AA. When the first openings OP1 are provided along the first direction Dx in the peripheral area GA, the pitch of the signal lines SL arranged adjacent to the first openings OP1 may be smaller than the pitch of the signal lines SL in the detection area AA.

This configuration makes a distance L2 between the gate lines GL1 in the peripheral area GA smaller than a distance L1 between the gate lines GL in the detection area AA, so that the first openings OP1 can be easily formed.

As illustrated in FIG. 18, in the detection device 1E, the first openings OP1 are provided so as to penetrate from the sealing film 210 to the first substrate 21 in the third direction Dz. In the present embodiment, the first openings OP1 are provided in areas overlapping peripheral area GA and not provided in areas overlapping the detection area AA. The side walls of the first openings OP1 are each covered with the resin film 55.

As a result, even when the detection device 1E is worn on the skin of the finger Fg or the like and used for a long time, the sweat generated from the finger Fg or the like is discharged through the first openings OP1 from the ventilation opening 221 to the outside. Therefore, even when the detection device 1E is worn on the skin of the finger Fg or the like and made adjacent to the skin, the moisture due to sweating is reduced, and the detection device 1E can be used for a long time.

The shape, number, and other factors of the first openings OP1 illustrated in FIGS. 16, 17, and 18 are merely exemplary and can be changed as appropriate. The first openings OP1 are not provided in some areas of the peripheral area GA, but are not limited to this configuration, and may be provided in the entire peripheral area GA, for example.

The detection device 1E according to the third embodiment can be combined with the detection device 1 according to the first embodiment. That is, the first openings OP and OP1 may be provided in the detection area AA and the peripheral area GA. In this case, the moisture due to sweating can be reduced more effectively.

### First Modification of Third Embodiment

FIG. 19 is a schematic sectional view of a detection device according to a first modification of the third embodiment. In the following description, the same components as those in either of the embodiments described above are denoted by the same reference numerals, and the description thereof will not be repeated.

As illustrated in FIG. 19, in a detection device 1F according to the first modification of the third embodiment, the side walls of the first openings OP1 are covered with the material 211 that is the same as the sealing film 210, compared with the detection device 1E according to the third embodiment.

As a result, the side walls of the first openings OP1 can be easily covered with only the sealing film 210, without using a member such as the resin film 55.

### Second Modification of Third Embodiment

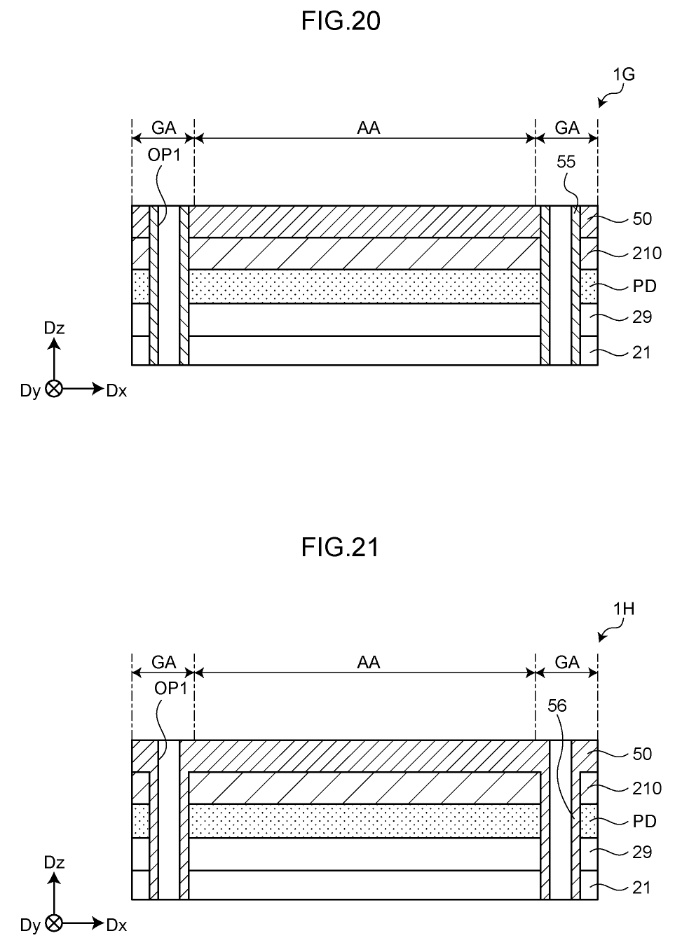

FIG. 20 is a schematic sectional view of a detection device according to a second modification of the third embodiment. In the following description, the same components as those in either of the embodiments described above are denoted by the same reference numerals, and the description thereof will not be repeated.

As illustrated in FIG. 20, in a detection device 1G according to the second modification of the third embodiment, the second substrate 50 is provided on the sealing film 210, compared with the detection device 1E according to the third embodiment. The first openings OP1 are provided so as to penetrate from the second substrate 50 to the first substrate 21 in the third direction Dz. The side walls of the first openings OP1 provided in the second substrate 50 are each covered with the resin film 55.

The configuration and operations of the detection device 1G according to the second modification of the third embodiment are substantially the same as those of the detection device 1E according to the third embodiment, and therefore, will not be described.

### Third Modification of Third Embodiment

FIG. 21 is a schematic sectional view of a detection device according to a third modification of the third embodiment. In the following description, the same components as those in either of the embodiments described above are denoted by the same reference numerals, and the description thereof will not be repeated.

As illustrated in FIG. 21, in a detection device 1H according to the third modification of the third embodiment, the second substrate 50 is provided on the sealing film 210, compared with the detection device 1E according to the third embodiment. The first openings OP1 are provided so as to penetrate from the second substrate 50 to the first substrate 21 in the third direction Dz. The side walls of the first opening OP1 are covered with the material 56 that is the same as the second substrate 50, instead of the resin film 55.

As a result, the side walls of the first openings OP1 can be easily covered with only the second substrate 50, without using a member such as the resin film 55.

### Fourth Embodiment

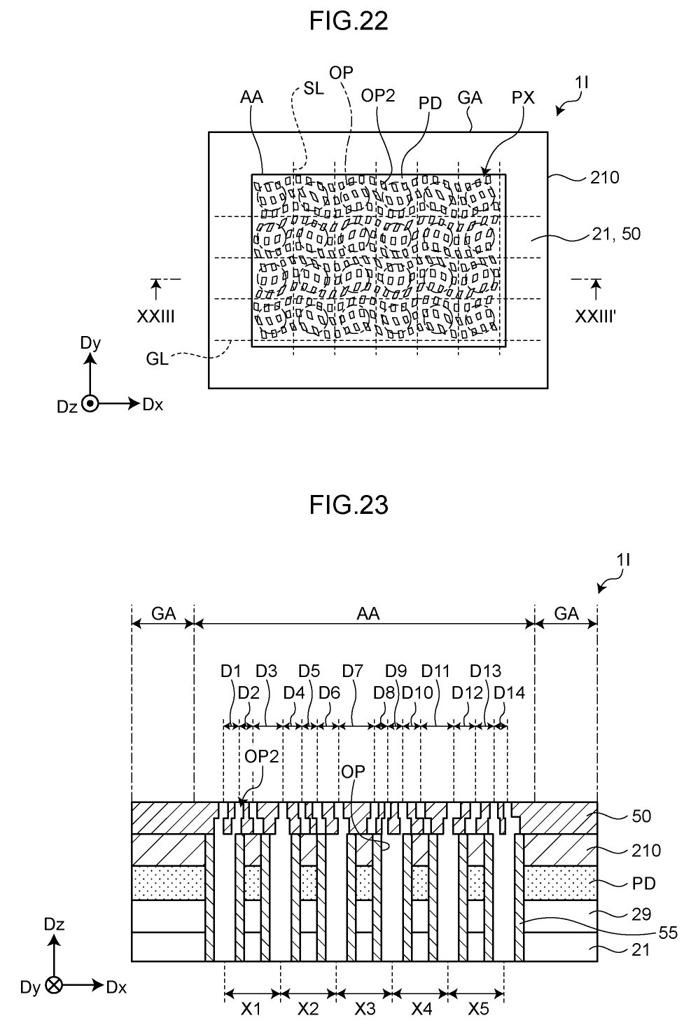

FIG. 22 is a plan view schematically illustrating the pixels, the first openings, and second openings of a detection device according to a fourth embodiment. FIG. 23 is a sectional view taken along XXIII-XXIII' in FIG. 22. In the following description, the same components as those in any one of the embodiments described above are denoted by the same reference numerals, and the description thereof will not be repeated.

As illustrated in FIG. 22, in a detection device 1I according to the fourth embodiment, the second substrate 50 is provided on the sealing film 210, compared with the detection device 1 according to the first embodiment. The second substrate 50 has a plurality of second openings OP2. The second substrate 50 is a mesh-shaped material in plan view. All or some of the second openings OP2 overlap the first opening OP. Each of the second openings OP2 is micrometer-sized.

As illustrated in FIG. 23, in the detection device 1I, the second substrate 50 has a single-layer structure. The second substrate 50 is not limited thereto, and may have a structure in which two or more layers are stacked.

Some of the centers of the second openings OP2 are misaligned with the centers of the first openings OP; the distances between the centers of the second openings OP2 are irregular; and the distances between the centers of the first openings OP are constant. Some of the centers of the second openings OP2 may be aligned with the centers of the first openings OP.

In detail, the magnitudes of center-to-center distances D1 to D14 of the second openings OP2 differ from one another, but the magnitudes of center-to-center distances X1 to X5 of the first openings OP are equal to one another.

With this configuration, even if the second substrate 50 cannot be provided with openings in the same way as the first openings OP, or even if the positions of the openings in the second substrate 50 cannot be aligned with those of the first openings OP, it is possible, by providing the second openings OP2, to discharge the sweat from the finger Fg or the like through the ventilation opening 221 to the outside via the second openings OP2 and the first openings OP. Therefore, even when the detection device 1I is worn on the skin of the finger Fg or the like and made adjacent to the skin, the moisture due to sweating is reduced, and the detection device 1I can be used for a long time.

### Manufacturing Method of Fifth Embodiment

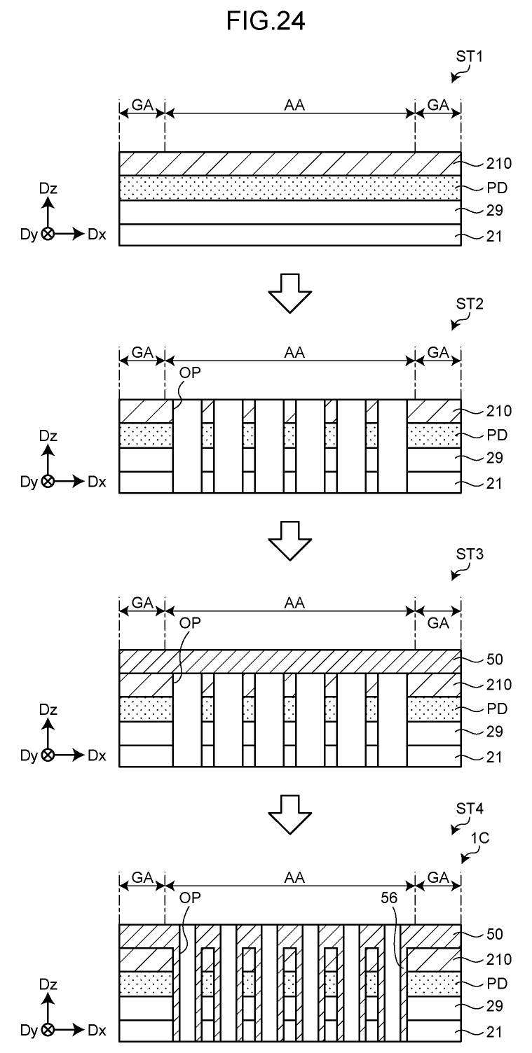

The following describes a manufacturing method according to a fifth embodiment. The manufacturing method of the fifth embodiment is a method for manufacturing the detection device 1C according to the third modification of the first embodiment. FIG. 24 is an explanatory diagram for explaining the manufacturing method of the fifth embodiment. In FIG. 24, the configuration of each layer is simplified for ease of understanding. In the following description, the same components as those in any one of the embodiments described above are denoted by the same reference numerals, and the description thereof will not be repeated.

As illustrated in FIG. 24, in the manufacturing method of the detection device 1C, the circuit forming layer 29, the organic photodiodes PD, and the sealing film 210 are stacked in this order on the first substrate 21 (Step ST1).

Then, the first openings OP are formed so as to penetrate from the sealing film 210 to the first substrate 21 in the third direction Dz (Step ST2).

Then, the second substrate 50 is stacked on the sealing film 210 (Step ST3). Finally, the second substrate 50 is heated (Step ST4). The processing method of the heating is a reflow process, for example.

The pressure in the first openings OP is lower than the pressure outside the second substrate 50, and the melting points of the sealing film 210 and the first substrate 21 are higher than the melting point of the second substrate 50. Therefore, at Step ST4, portions of the second substrate 50 that overlap the first openings OP are deformed while melting due to a difference in pressure between the second substrate 50 and the first openings OP. As a result, the portions of the second substrate 50 that overlap the first openings OP are deformed in the third direction Dz along inner walls of holes toward the first substrate 21 (Step ST4). The side walls of the first openings OP are then covered with the same material 56 as the second substrate 50, and the material 56 hardens after the heating ends. The detection device 1C according to the third modification of the first embodiment is manufactured by the above processing.

In the manufacturing method of the detection device 1C according to the third modification of the first embodiment, the second substrate 50 is easily formed from one plate, the holes are formed in the portions of the second substrate 50 that overlap the first openings OP, and the side walls of the first openings OP are covered. The manufacturing method illustrated in FIG. 24 is merely exemplary, and can be changed as appropriate.

While the preferred embodiments have been described above, the present disclosure is not limited to the embodiments described above. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications thereof described above.

What is claimed is:

1. A detection device comprising:
a first substrate;
a plurality of organic photodiodes each comprising a lower electrode, a lower buffer layer, an active layer, an upper buffer layer, and an upper electrode, which are stacked on the first substrate;
a detection area in which the organic photodiodes are arranged;
a sealing film that covers the detection area; and
a plurality of first openings that are provided so as to penetrate at least from the sealing film to the first substrate.

2. The detection device according to claim 1, further comprising a second substrate that covers the sealing film, wherein
the first openings are provided so as to penetrate from the second substrate to the first substrate.

3. The detection device according to claim 1, comprising a plurality of sensor pixels that are arranged in a matrix having a row-column configuration in the detection area and comprise the respective organic photodiodes, wherein
each of the sensor pixels is surrounded by adjacent gate lines and adjacent signal lines, and
the first openings are provided in areas overlapping the sensor pixels and are arranged in a matrix having a row-column configuration in the detection area.

4. The detection device according to claim 1, wherein areas of the first openings are substantially equal to one another.

5. The detection device according to claim 1, wherein each of the first openings is provided in an area surrounded by adjacent gate lines and adjacent signal lines.

6. The detection device according to claim 1, wherein the first openings are provided in areas in which the lower electrode is not disposed in plan view.

7. The detection device according to claim 1, wherein side walls of the first openings are covered with the same material as the sealing film.

8. The detection device according to claim 2, wherein side walls of the first openings are covered with the same material as the second substrate.

9. The detection device according to claim 1, comprising a peripheral area adjacent to the detection area, wherein
the first openings are provided in the peripheral area.

10. The detection device according to claim 3, comprising:
a peripheral area adjacent to the detection area; and
wiring lines coupled to the gate lines or the signal lines, wherein
the first openings are provided in the peripheral area, and
a pitch of the wiring lines arranged adjacent to the first openings in the peripheral area is smaller than a pitch of the gate lines or the signal lines in the detection area.

11. The detection device according to claim 9, wherein side walls of the first openings are covered with the same material as the sealing film.

12. The detection device according to claim 2, comprising a peripheral area adjacent to the detection area, wherein
the first openings are provided in the peripheral area, and
side walls of the first openings are covered with the same material as the second substrate.

13. The detection device according to claim 1, further comprising a second substrate provided on the sealing film, wherein
the second substrate has a mesh shape.

14. A method for manufacturing a detection device, the method comprising:
stacking a circuit forming layer, an organic photodiode, and a sealing film that covers the organic photodiode on one surface of a first substrate in the order as listed;
forming, after the stacking, a first opening so as to penetrate from the sealing film to the first substrate in a direction orthogonal to the first substrate;
stacking a second substrate on the sealing film after the forming; and
heating the second substrate after the stacking the second substrate.

15. The method according to claim 14, wherein
in the heating:
pressure in the first openings is lower than pressure outside the second substrate, and
melting points of the sealing film and the first substrate are higher than a melting point of the second substrate.

16. A detection device comprising:
a first substrate;
a plurality of photodiodes each comprising a lower electrode, a lower buffer layer, an active layer, and an upper buffer layer that doubles as an upper electrode, which are stacked on the first substrate;
a detection area in which the photodiodes are arranged;
a sealing film that covers the detection area; and
a plurality of first openings that are provided so as to penetrate at least from the sealing film to the first substrate.

17. A detection device comprising:
a first substrate;
a plurality of photodiodes each comprising a lower electrode, a lower buffer layer, an active layer, an upper buffer layer, and an upper electrode that doubles as a sealing film, which are stacked on the first substrate;
a detection area in which the photodiodes are arranged; and
a plurality of first openings that are provided so as to penetrate at least from the upper electrode to the first substrate.

18. A method for manufacturing a detection device, the method comprising:
stacking a circuit forming layer, a photodiode, and a sealing film that covers the photodiode on one surface of a first substrate in the order as listed;
forming, after the stacking, a first opening so as to penetrate from the sealing film to the first substrate in a direction orthogonal to the first substrate;
stacking a second substrate on the sealing film after the forming; and
heating the second substrate after the stacking the second substrate.
